(19) United States
(12) Patent Application Publication
(10) Pub. No.: US 2004/0254541 A1
(43) Pub. Date: Dec. 16, 2004

(54) Non-sharp vascular infusion cannula

(76) Inventors: K. C. Wong, Taipei (TW); Ming-Hsiung Tsai, Taichung (TW)

Correspondence Address: Parsons Behle & Latimer, Suite 1800, 201 South Main Street, P. O. Box 45898, Salt Lake City, UT, 84111, US

(21) Appl. No.: 10/780,508

(22) Filed: Feb. 17, 2004

Related U.S. Application Data
(60) Provisional application No. 60/464,260, filed on Apr. 21, 2003.

(51) Int. Cl. A61M 5/00

(52) U.S. Cl. 604/239; 604/912

(57) ABSTRACT

A non-sharp vascular infusion device and related method are disclosed. The non-sharp vascular infusion device permits medicine to be introduced into an IV assembly without creating a danger to healthcare personnel.

### CROSS-REFERENCE TO RELATED APPLICATIONS

This application claims the benefit of U.S. Provisional Patent Application Ser. No. 60/464,260 filed Apr. 21, 2003, and which is hereby incorporated by reference in its entirety.

### BACKGROUND

In the field of medicine, there are many instances when a health care provider will need to aspirate medicine from a medicine vial or to inject medicine into an intravenous fluid assembly to permit it to flow into a patient. In the past, typically sharp metal needles were used for such tasks presents a danger to both the health care provider and the patient. During use, a sharp can inadvertently impale a patient or a health care provider, potentially transmitting disease. If a sharp is not disposed of properly, it may inadvertently impale housekeeping staff, raising the same disease concerns.

There is a need for a simple, inexpensive and disposable non-sharp vascular infusion cannula. A user may choose to use the cannula to aspirate medicine from a medicine vial once or repeatedly, and to inject medicine into an I.V. apparatus once or repeatedly.

### SUMMARY

In the various embodiments disclosed, a non-sharp cannula is provided. The non-sharp cannula may have a square, blunt, tapered or rounded distal end, and a proximal end configured for attachment to a syringe or other fluid source. The non-sharp cannula can have an elongate body with a hollow fluid passageway in its interior for transporting fluid from either of the proximal or distal end to the other. The non-sharp cannula can have a fluid exit orifice in the vicinity of the cannula distal end, but located to the side of the cannula distal end so that fluid that exits the exit orifice may not be traveling parallel to the path of fluid that is travelling through the hollow fluid passageway of the cannula interior. Use of a side exit orifice allows the cannula to penetrate a soft rubber or plastic membrane, such as would be found on a medicine vial or an intravenous fluid assembly, without cutting a section from the fluid membrane.

As desired the non-sharp vascular infusion cannula may be configured with a mechanical connection for attaching to a syringe. Example connections include a luer lock, a tapered friction fit, and other known mechanical attachments. In some embodiments it may be desired to include an optional one-way valve or seal within the non-sharp cannula so that fluid may only flow in one direction through the cannula. In other embodiments, such a one-way valve or seal may be omitted.

The non-sharp vascular infusion cannula may be used in conjunction with a syringe, or it may be used in conjunction with any one-time, repetitive or continuous drug infusion process, such as may be employed in the field of anesthesiology. It may also be permanently placed into a medicine vial to permit multiple syringes to aspirate medicine from the vial.

The non-sharp vascular infusion cannula is user friendly in that it permits all medical care providers with a medicine aspiration and infusion device that will not inadvertently impale them or the patient. The cannula is not a sharp, so it may be disposed of with ordinary refuse, rather than being placed in a sharps container for more expensive disposal. If housekeeping staff come into contact with the non-sharp vascular infusion cannula, they are not placed at risk of injury or of contracting disease. If made of modern materials, the non-sharp vascular infusion cannula is environmentally friendly in that it is recyclable. It is also inexpensive to manufacture and therefore presents an economical disposable solution to many problems that heretofore only expensive devices attempted to solve. Finally, the non-sharp vascular infusion cannula is a non-sharp, it will not be attractive for acquisition by substance abusers who may be seeking a sharp for self-injection.

The non-sharp vascular infusion cannula may be made from any appropriate materials, but at the present time it is expected that the most desirable material may be plastic. The non-sharp vascular infusion cannula presents a physical form that is highly unlikely to injure either the user or the patient. The non-sharp vascular infusion cannula can be used to penetrate a multiple use drug vial for drug aspiration, it can be used to penetrate the infusion port of an infusion tube or intravenous assembly to inject medicine therein, and it can be used to penetrate the drug adding port of an I.V. bag to inject medicine therein. Other objects, features and advantages of the non-sharp vascular infusion cannula will become apparent to the reader upon reading the specification in light of the appended drawings.

### DETAILED DESCRIPTION

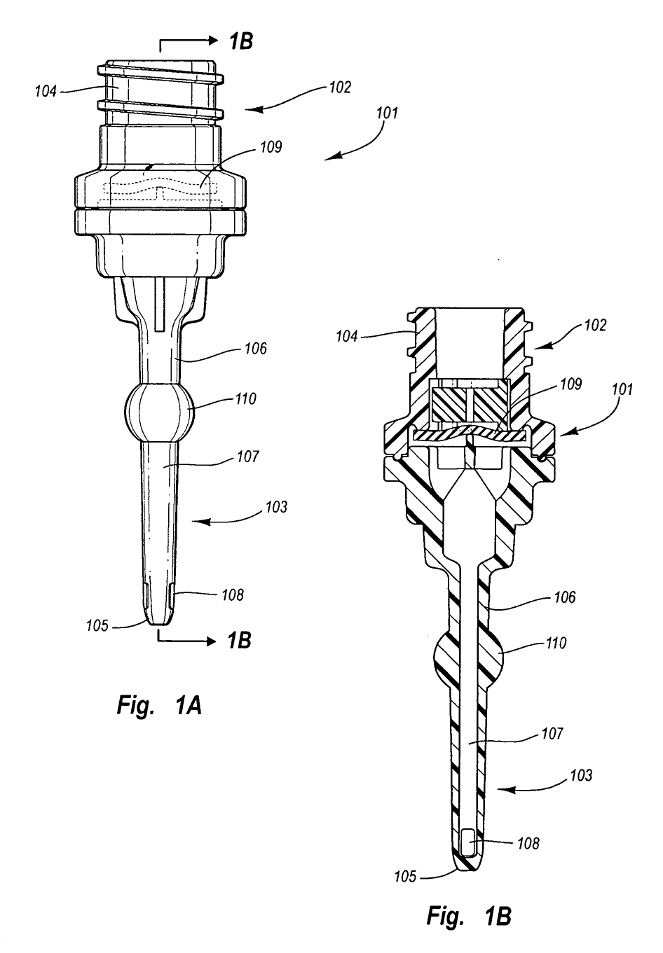

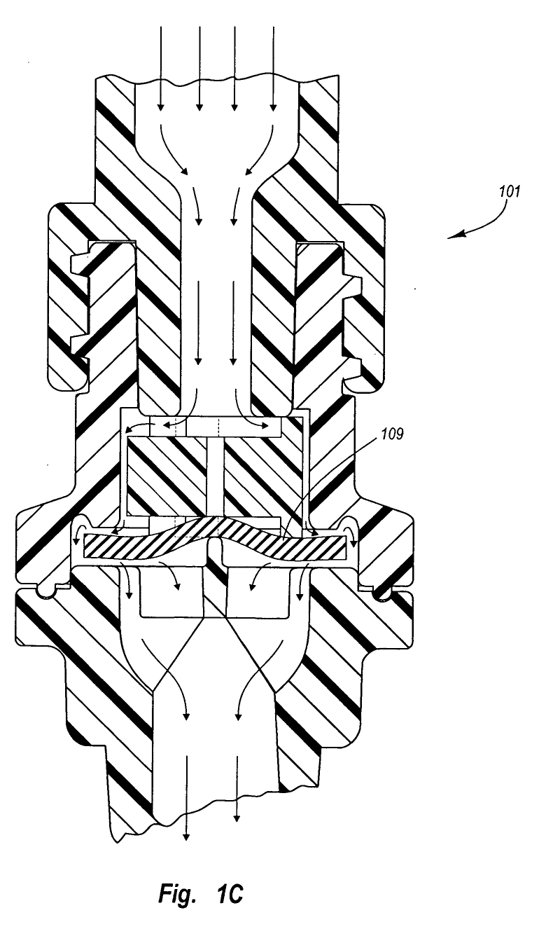

Referring to FIG. 1A, a side view of a non-sharp vascular infusion cannula 101 is depicted. FIG. 1 B depicts a cross-sectional view of the non-sharp fluid cannula of FIG. 1A. The non-sharp vascular infusion cannula 101 includes a proximal end 102 and a distal end 103. The proximal end 102 may include a mechanical connection 104 for attaching to a syringe or other fluid source. The mechanical connection 104 may be a threaded fitting such as a luer lock (as depicted), a tapered receptacle for friction fit with a syringe tip or other fluid source protuberance, or any other mechanical fitting. The distal end 103 of the non-sharp vascular infusion cannula 101 has a non-sharp tip 105 which may be square, blunt, tapered or rounded. The non-sharp tip will not impale human skin when placed thereagainst with ordinary finger pressure, thereby avoiding inadvertent impalement and the attendant health concerns.

Between the proximal end 102 and the distal end 103 of the non-sharp fluid cannula 101 there is an elongate body 106 with a hollow fluid passageway 107 in its interior for transporting fluid from either of the proximal or distal end to the other. The non-sharp cannula 101 has one or more fluid exit orifices 108 that may be located along the elongate body 106 and hollow fluid passageway 107, such as in the vicinity of the cannula distal end. The exit orifices may be located to the side of the cannula distal end so that fluid that exits the exit orifice may not be traveling parallel to the path of fluid that is travelling through the hollow fluid passageway of the cannula interior. Use of a side exit orifice allows the cannula to penetrate a soft rubber or plastic membrane, such as would be found on a medicine vial or an intravenous fluid assembly, without cutting a section from the fluid membrane.

In some embodiments it may be desired to include a one-way valve or seal 109 within the non-sharp cannula so that fluid may only flow in one direction through the cannula. In the example shown, a one-way valve is employed so that fluid may flow from the proximal to the distal end of the non-sharp vascular infusion cannula, but not the reverse. More detail concerning the example valve 109 is presented in FIG. 1C. Many valves and seals are known in the field of hydraulics which may be employed. Alternatively, the non-sharp vascular infusion cannula may be used without a valve or seal.

The non-sharp vascular infusion cannula 101 has a radial bulge, radial protuberance, obstruction or obturation 110 located along its body 106. The radial bulge serves two purposes. First, when the non-sharp vascular infusion cannula penetrates the membrane of a medicine vial or a vascular infusion cannula port, if desired, the radial bulge may be pushed past such membrane in order to anchor the non-sharp vascular infusion cannula in position for multiple uses. Second, if it is desired to remove the non-sharp vascular infusion cannula from a membrane such as the membrane on a medicine vial or the membrane on a vascular infusion cannula port after one use, then the non-sharp vascular infusion cannula may be used to penetrate the membrane up to but not past the radial bulge 110 in order for the non-sharp vascular infusion cannula to be easily removed from the membrane.

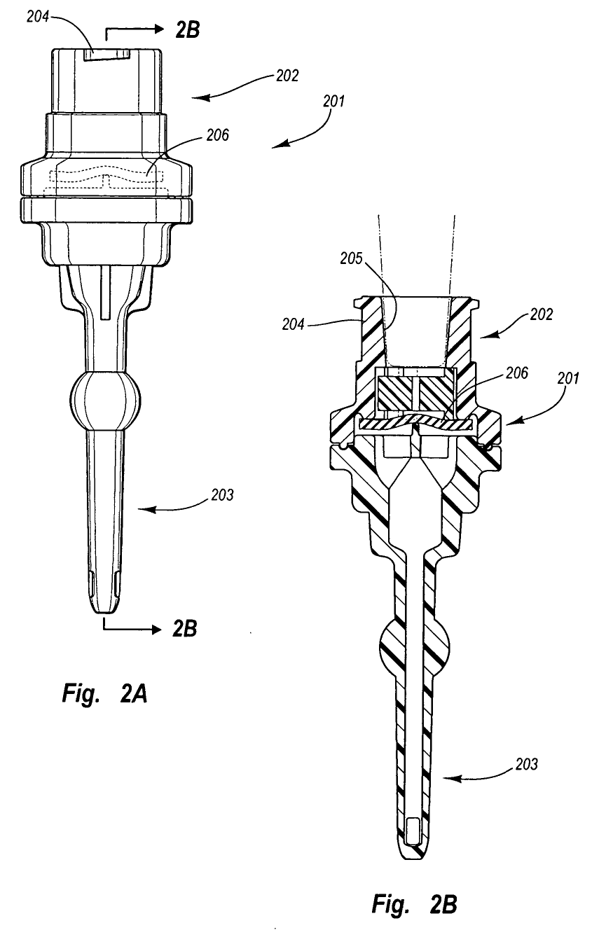

Referring to FIGS. 2A and 2B, another embodiment of a non-sharp fluid cannula 201 is depicted. The non-sharp vascular infusion cannula 201 includes a proximal end 202 and a distal end 203. The proximal end 202 includes a receptacle 204 for insertion of a syringe tip or other projection therein for forcing fluid into the cannula or for receiving fluid from the fluid cannula. The receptacle 204 may be presented with tapered cylindrical walls 205 if desired for creating a friction fit with the structure placed into the receptacle. A valve or seal 206 may be provided within the non-sharp vascular infusion cannula, or the valve or seal may be omitted and a hollow passageway substituted therefor.

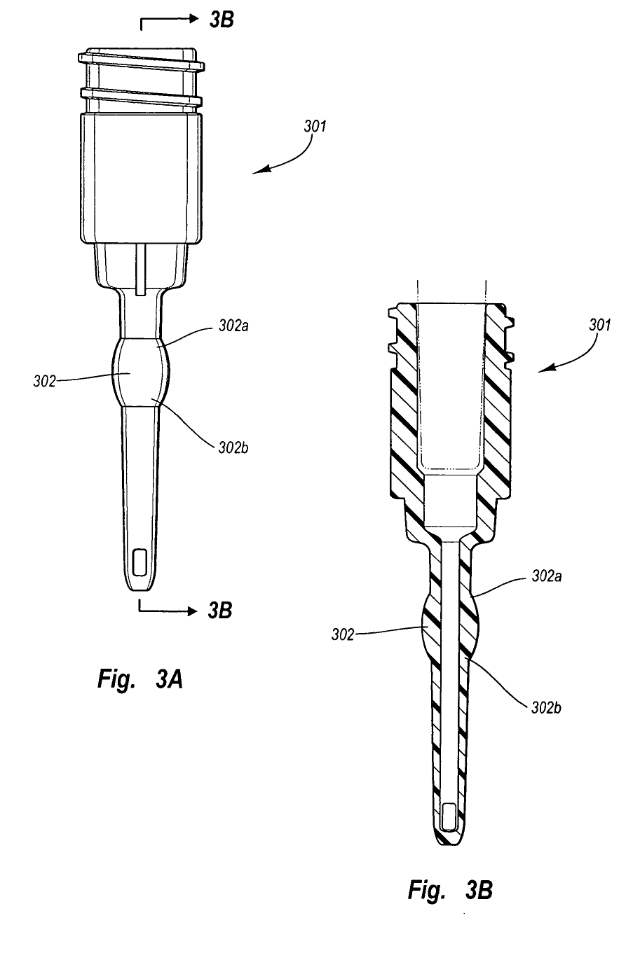

Referring to FIGS. 3A and 3B, another embodiment of a non-sharp fluid cannula 301 is depicted. In this embodiment, the radial bulge 302 is depicted as being partially oval or elliptical, rather than spherical as in FIGS. 1A and 2A. If desired, it is also possible to present a radial bulge 302 which has a steeper or sharper curvature or radius at its proximal side 302a than at its distal side 302b. Using a mild curvature or radius or slope on the distal side of the radial bulge will allow it to easily penetrate a membrane of a vascular infusion cannula port or medicine vial. Using a steeper or sharper curvature, radius or slope on the proximal side of the radial bulge will tend to make it difficult to withdraw the non-sharp fluid cannula from a medicine vial or vascular infusion cannula port once the non-sharp vascular infusion cannula has been inserted into the membrane of the medicine vial or vascular infusion cannula port past the radial bulge. Such a configuration is desirable when the non-sharp vascular infusion cannula is to be placed into a medicine vial or vascular infusion cannula port to be fixed there for multiple uses. The reader should note that the non-sharp vascular infusion cannula of FIGS. 3A and 3B includes both a luer lock and a tapered friction receptacle for fitting to a syringe or other appropriate device.

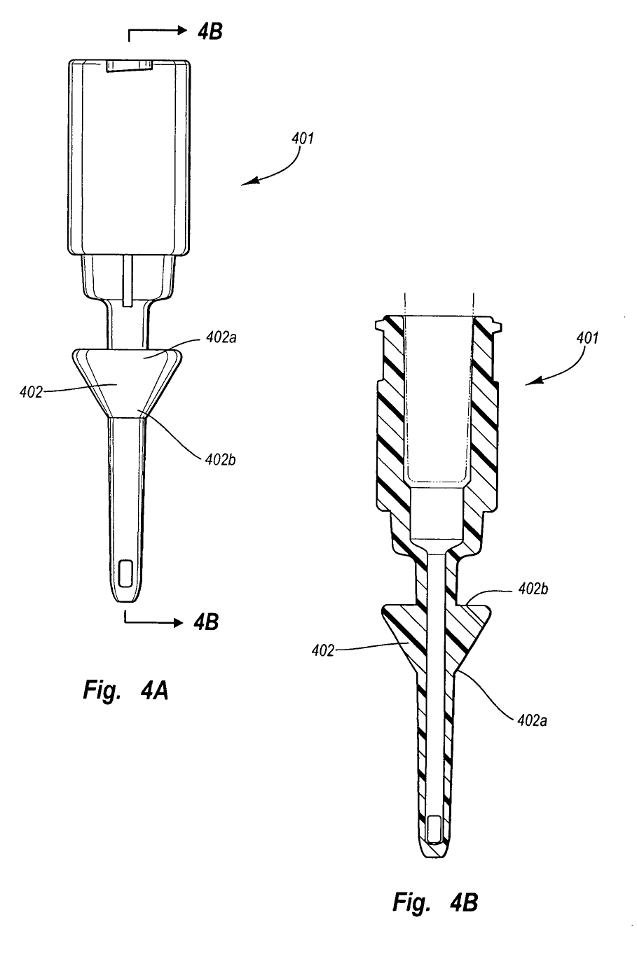

Referring to FIGS. 4A and 4B, an alternative radial bulge 402 on a non-sharp vascular infusion cannula 401 is depicted. The radial bulge 402 depicted is generally triangular in cross section or conical in 3-D so that it presents a relatively gentle slope at its distal side 402a for penetrating a membrane of a medicine vial or vascular infusion cannula port, but it presents a flat surface 402b at the proximal side 402b of the radial bulge 402 so that withdrawing the non-sharp vascular infusion cannula from a membrane once it has been inserted past the radial bulge 402 will be very difficult or impossible.

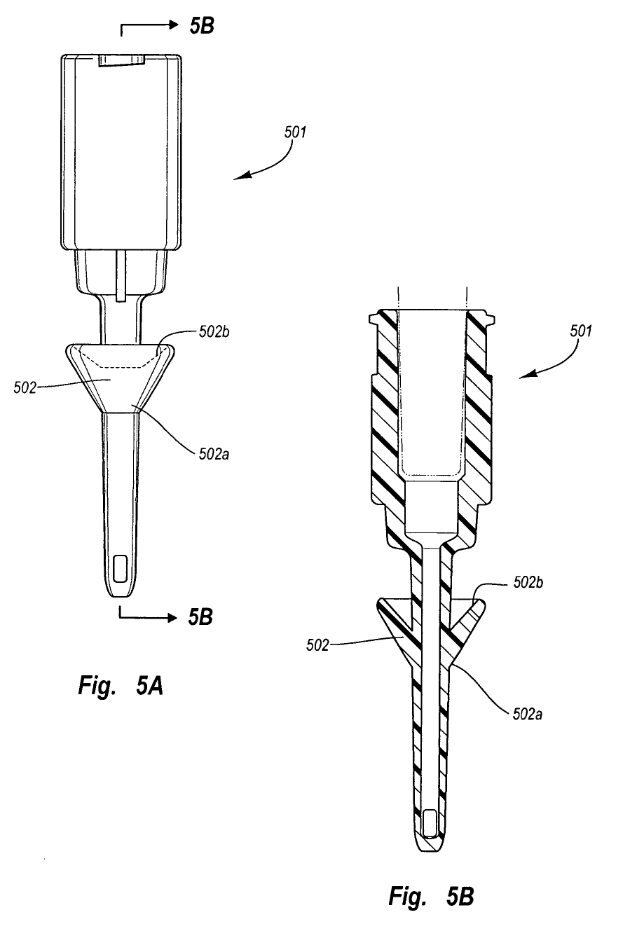

Referring to FIGS. 5A and 5B, an alternative radial bulge 502 on a non-sharp vascular infusion cannula 501 is depicted. The radial bulge 502 depicted has a rebated or hooked proximal side 502b, but has a relatively gentle slope at its distal side 502a for penetrating a membrane of a medicine vial or vascular infusion cannula port. This configuration makes it easy to insert the non-sharp vascular infusion cannula through a membrane past the radial bulge 502, but difficult or impossible to withdraw the non-sharp vascular infusion cannula from the membrane.

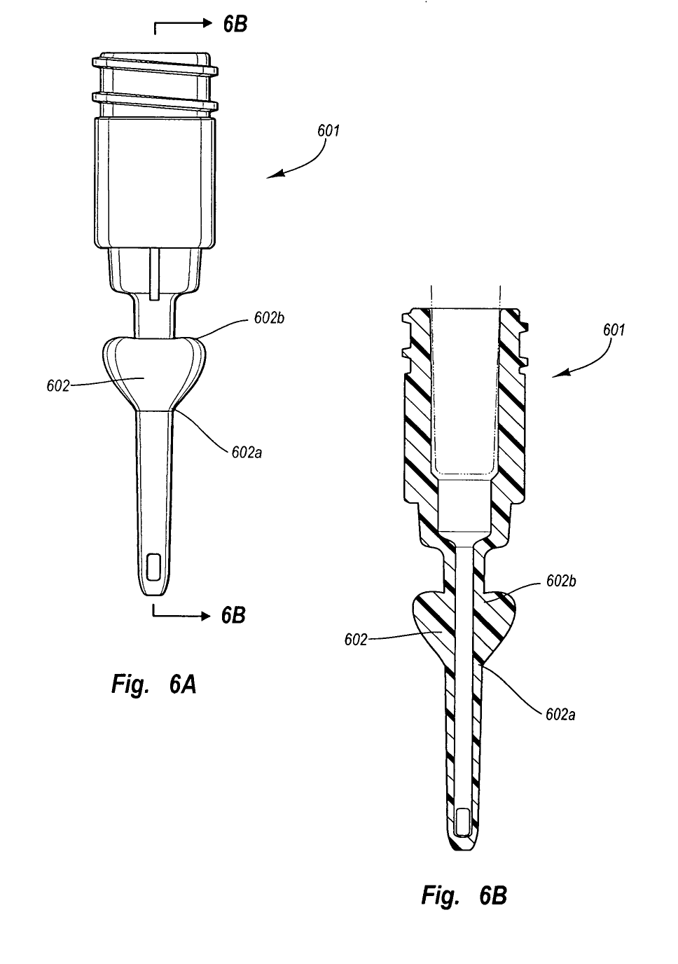

Referring to FIGS. 6A and 6B, an alternative radial bulge 602 on a non-sharp vascular infusion cannula 601 is depicted. The radial bulge 602 depicted is heart-shaped, so it has a gentle slope, curvature or radius on its distal side 602a for easy insertion into a membrane of a medine vial or vascular infusion port. But it has a steep curvature or radius on its proximal side 602b to make it very difficult or impossible to withdraw from the membrane.

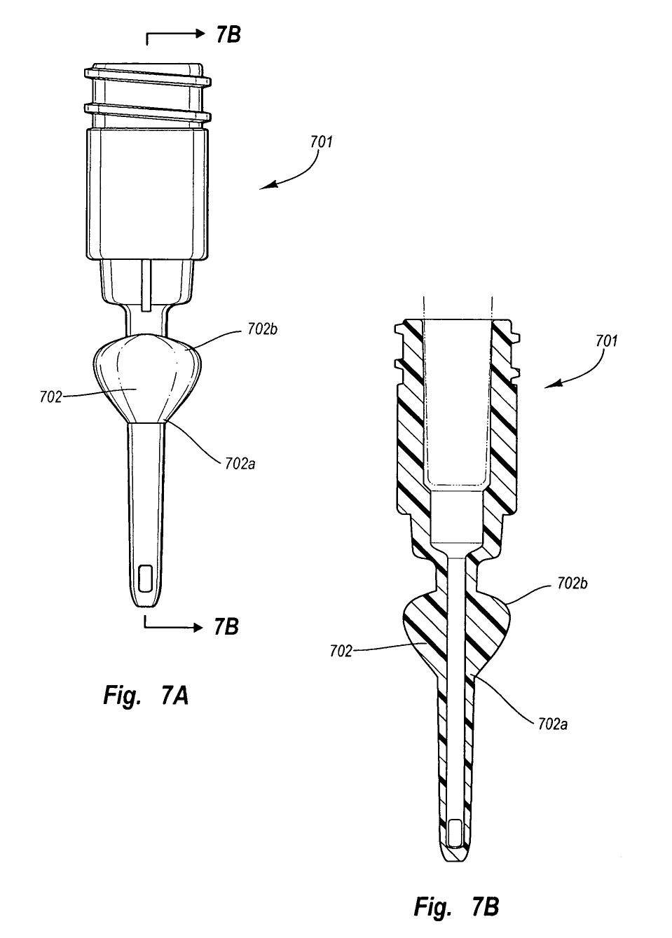

Referring to FIGS. 7A and 7B, an alternative radial bulge 702 on a non-sharp vascular infusion cannula 701 is depicted. The radial bulge 702 depicted is turnip or top-shaped, so it has a gentle slope, curvature or radius on its distal side 702a for easy insertion into a membrane of a medine vial or vascular infusion cannula port. But it has a steep curvature or radius on its proximal side 702b to make it very difficult or impossible to withdraw from the membrane.

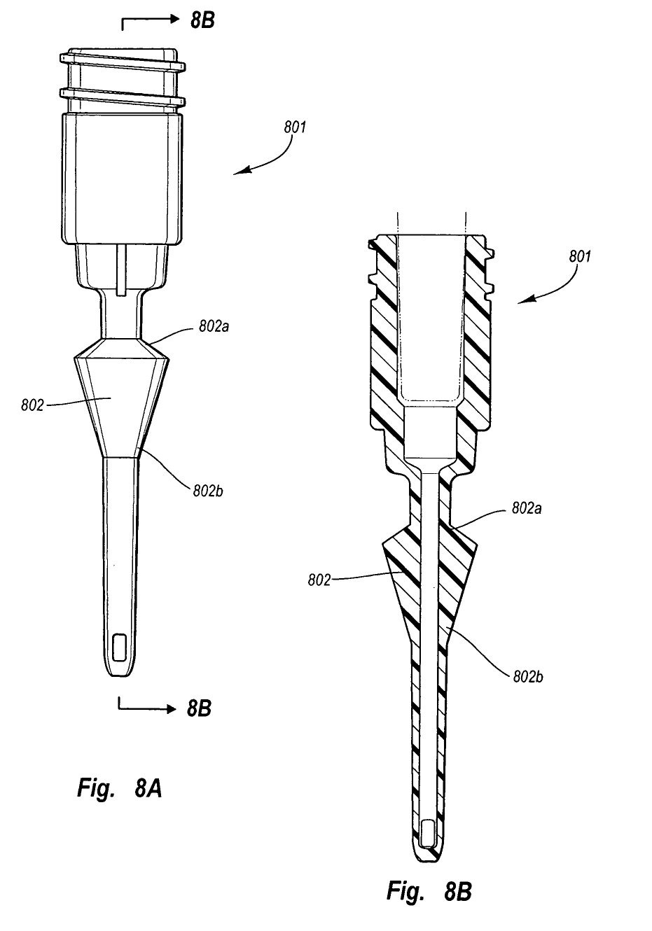

Referring to FIGS. 8A and 8B, an alternative radial bulge 802 on a non-sharp vascular infusion cannula 801 is depicted. The radial bulge 802 depicted is diamond-shaped in cross section. It has a gentle slope on its distal side 802a for easy insertion into a membrane of a medicine vial or vascular infusion cannula port. But it has a radius on its proximal side 802b to make it very difficult or impossible to withdraw from the membrane.

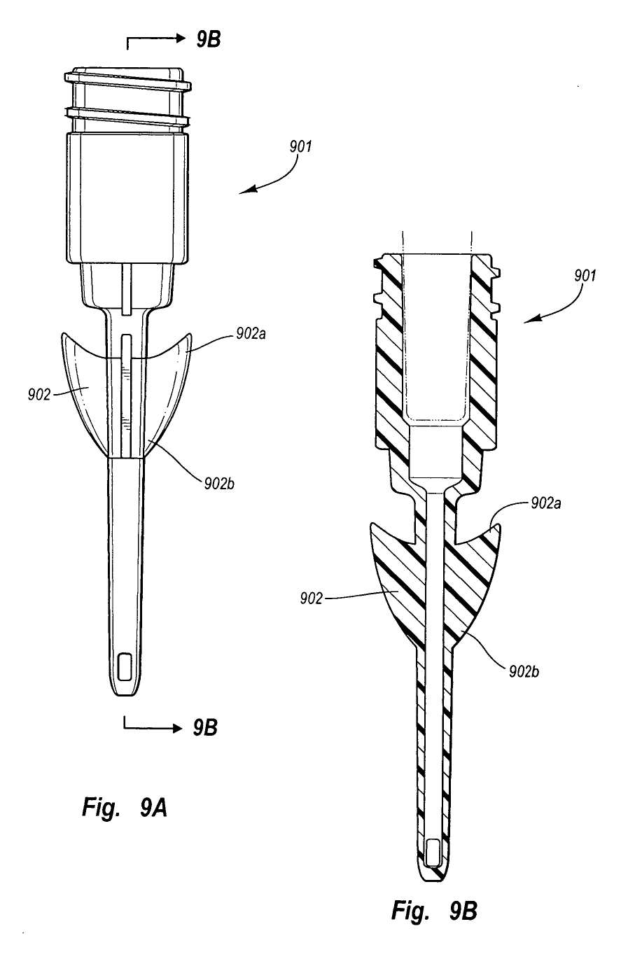

Referring to FIGS. 9A and 9B, an alternative radial bulge 902 on a non-sharp vascular infusion cannula 901 is depicted. The radial bulge 902 depicted is presented in a fish hook configuration (in cross section). It has a gentle radius or curvature on its distal side 902a for easy insertion into a membrane of a medicine vial or vascular infusion cannula port. But it has a reverse cut radius on its proximal side 902b that presents a sharp or barbed outer rim to make it very difficult or impossible to withdraw from the membrane.

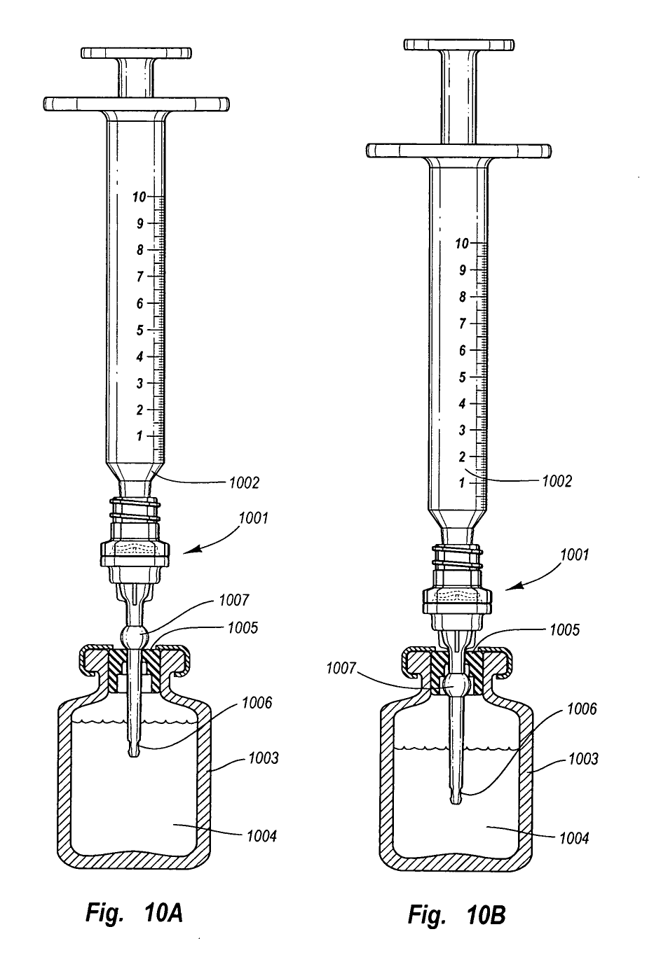

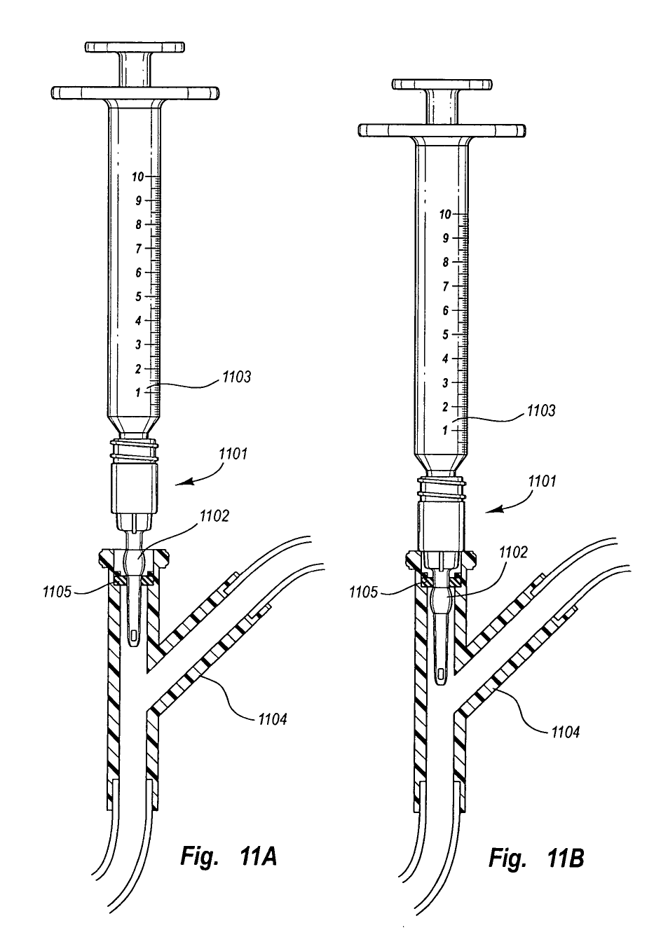

Referring to FIG. 10A, a non-sharp vascular infusion cannula 1001 is depicted attached to a syringe 1002. This assembly is being used to aspirate medicine 1004 from a medicine vial 1003. The distal end of the non-sharp vascular infusion cannula has been thrust deep enough into the medicine vial so that the orifice 1006 of the cannula is in the medicine 1004 in order aspirate the medicine 1004 through the orifice 1006 through the cannula 1001 and into the interior of the syringe 1002. In FIG. 10A, the cannula has been inserted through the rubber membrane 1005 of the medicine vial up to but not beyond the radial bulge 1007 of the cannula. This permits aspiration of medicine from the vial and into the syringe and withdrawal of the cannula from the medicine vial after the aspiration is complete. Thereafter, the syringe with non-sharp vascular infusion cannula may be transported to an I.V. apparatus in order to inject the aspirated medicine therein.

Referring to FIG. 10B, the same configuration is depicted, but the cannula has been inserted deeply enough into the medicine vial so that the radial bulge 1007 of the cannula has been projected past the rubber membrane of the medicine vial. The shape of the radial bulge 1007 will present significant resistance to any attempt to remove the cannula from the medicine vial. Thus, the non-sharp vascular infusion cannula 1001 may be left in the medicine vial for multiple uses, while the syringe can be removed from the non-sharp fluid cannula and another syringe affixed to it for aspiration of additional medicine from the medicine vial. This allows repetitive use of the non-sharp vascular infusion cannula with various syringes.

Referring to FIG. 11A, a non-sharp vascular infusion cannula 1101 is depicted attached to a syringe 1103 for injecting medicine into a vascular infusion cannula port 1104 of an intravenous assembly. The vascular infusion cannula port 1104 includes a membrane 1105 which may be penetrated by the non-sharp vascular infusion cannula. The cannula is depicted as having penetrated the membrane up to but not past the radial bulge 1102 so that the non-sharp vascular infusion cannula may be easily removed from the infusion port.

Referring to FIG. 11B, the same configuration is depicted, but the non-sharp vascular infusion cannula 1101 has been inserted into the infusion port 1104 so deeply that the radial bulge 1102 of the non-sharp vascular infusion cannula 1101 projects beyond the membrane 1105. If an attempt is made to remove the non-sharp vascular infusion cannula from the infusion port 1104 past the membrane 1105, it will be difficult or impossible to do so because of the size and shape of the radial bulge 1102.

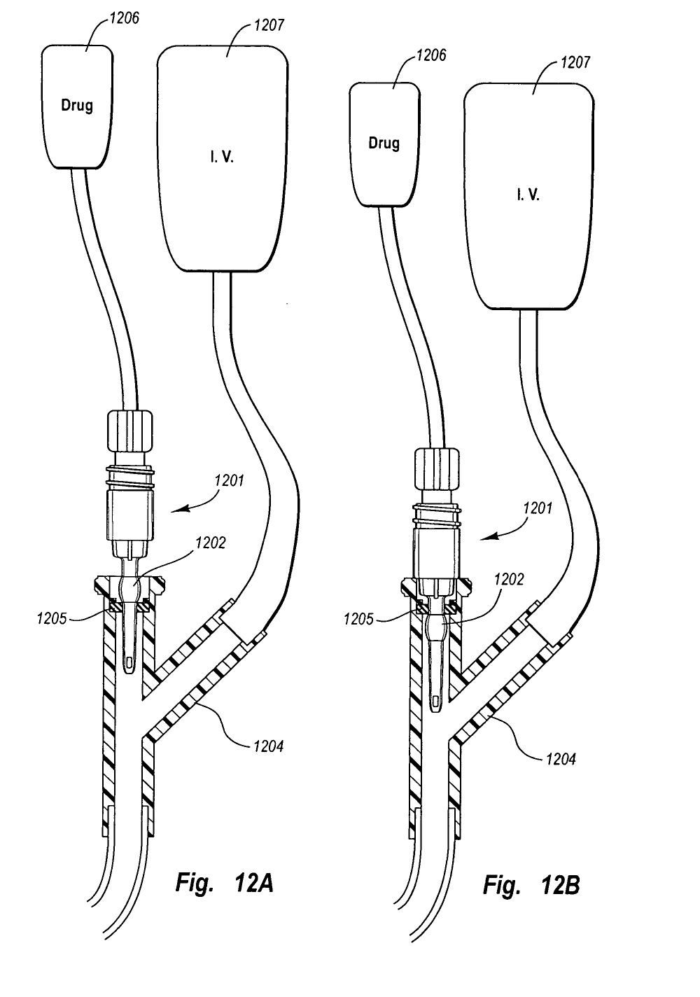

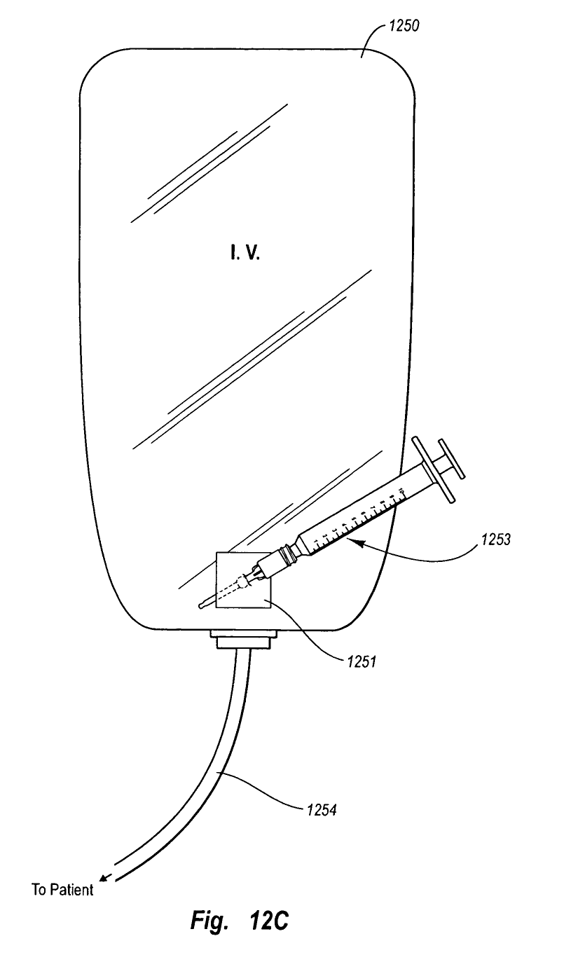

Referring to FIG. 12A, a non-sharp vascular infusion cannula is shown being used for continuous infusion of medicine in an I.V. environment. The non-sharp fluid cannula 1201 is projecting through a membrane 1205 of an infusion port 1204 of an I.V. arrangement. I.V. fluid 1207 passes through the infusion port 1204, and a drug or medicine 1206 also passes through the infusion port to a patient. The non-sharp vascular infusion cannula projects through the membrane 1205 up to but not beyond the radial bulge 1202 of the non-sharp vascular infusion cannula so that the cannula may be later removed.

Referring to FIG. 12B, the same configuration is depicted, but the radial bulge 1202 has been projected beyond the membrane 1205 so that the non-sharp vascular infusion cannula cannot easily be removed from the infusion port. In such an arrangement, drug bags may be replaced as often as desired without replacing or moving the non-sharp vascular infusion cannula and without exposing medical care providers to use of a sharp.

Referring to FIG. 12C, use of a non-sharp vascular infusion cannula attached to a syringe 1253 is depicted for injecting a medicine or drug directly into an I.V. bag 1250 through a membrane 1251 to that the drug or medicine will travel with the I.V. fluid through passageway 1254 to a patient.

Although the distal end of the non-sharp vascular infusion cannula has been described as being blunt, round, or radiused, its lack of a sharp tip does not mean that it is completely incapable of impalement. The tip of the non-sharp vascular infusion cannula usually will not be sharp enough to impale human skin under pressures normally applied by fingers or the human hand, but it will be capable of penetrating the softer membranes of medicine vials and vascular infusion cannula ports. Placement of the fluid exit orifice to the side of the tip of the non-sharp vascular infusion cannula improves the ability of the cannula to impale a soft rubber or plastic membrane, but the exit orifice may be placed at any desired location.

The non-sharp vascular infusion cannula may be made from any appropriate materials, but at the present time it is expected that the most desirable material will be plastic. The non-sharp vascular infusion cannula presents a physical form that is highly unlikely to injure either the user or the patient.

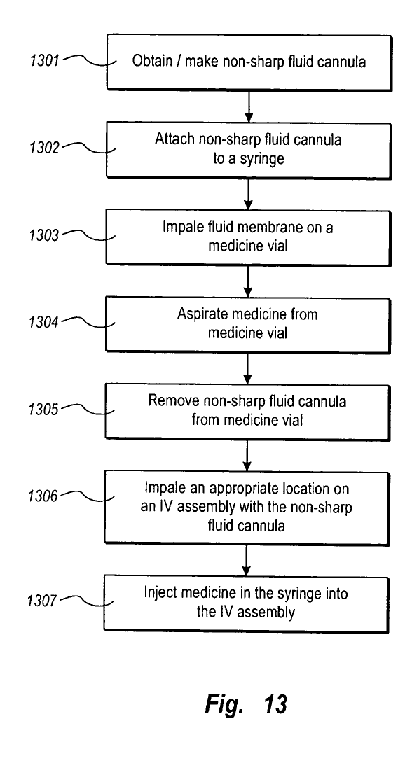

Referring to FIG. 13, a method for using a non-sharp vascular infusion cannula is depicted as a series of steps. First, the user should obtain or make a non-sharp vascular infusion cannula 1301. The user should attach the non-sharp vascular infusion cannula to a syringe 1302 and impale a fluid membrane on a medicine vial 1303 with the non-sharp vascular infusion cannula. Then the non-sharp vascular infusion cannula should be used to aspirate medicine from the medicine vial 1304, and the non-sharp vascular infusion cannula should be removed from the medicine vial 1305. Medicine is at that time contained within the syringe. Next, the non-sharp vascular infusion cannula should be used to impale an appropriate location on an I.V. assembly 1306, and medicine should then be injected from the syringe through the non-sharp vascular infusion cannula into the I.V. assembly 1307 where it can flow to a patient. Then either the non-sharp vascular infusion cannula and the syringe may be removed from the I.V. assembly, or the non-sharp vascular infusion cannula can be left in place on the I.V. assembly but the syringe removed so that medicine may later be introduced into the I.V. assembly through the non-sharp vascular infusion cannula.

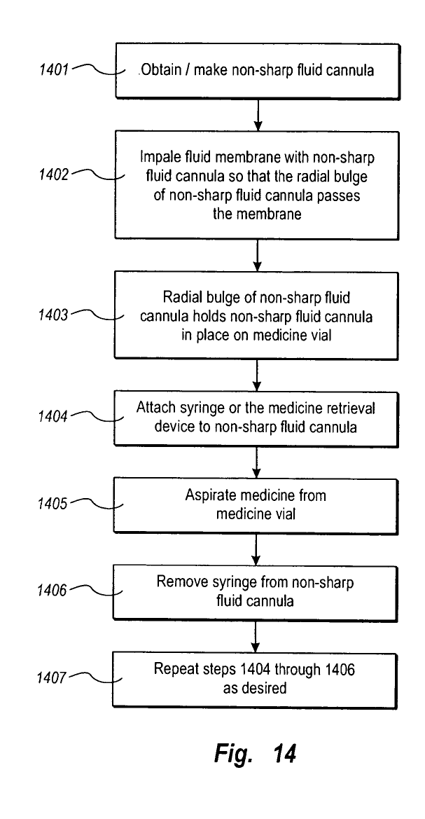

Referring to FIG. 14, another method for using a non-sharp vascular infusion cannula is depicted. First, a non-sharp vascular infusion cannula must be made or obtained 1401. Then the non-sharp vascular infusion cannula is used to impale a fluid membrane 1402 so that the radial bulge of the non-sharp vascular infusion cannula passes through the membrane, thereby anchoring the non-sharp vascular infusion cannula in place on the fluid membrane. At that point, the radial bulge of the non-sharp fluid cannula holds the non-sharp vascular infusion cannula in place at the membrane 1403. The membrane can be located at a medicine vial or other location. Then a syringe or medicine retrieval device is attached to the non-sharp vascular infusion cannula 1404. Medicine is aspirated past the membrane through the non-sharp vascular infusion cannula 1405 and the syringe can then be removed from the non-sharp vascular infusion cannula with the medicine located within it. Steps 1404 through 1406 can be repeated as desired to that the non-sharp vascular infusion cannula is used multiple times to dispense medicine.

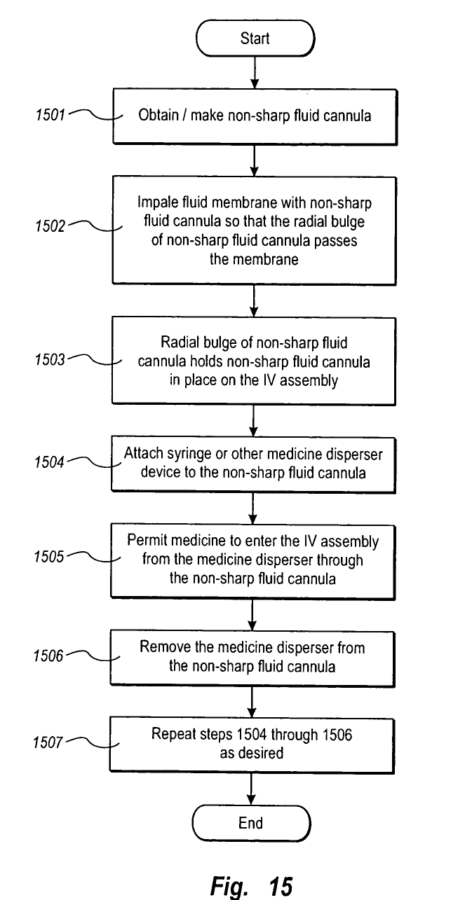

Referring to FIG. 15, first a user obtains or makes a non-sharp vascular infusion cannula 1501. Then the non-sharp vascular infusion cannula 1502 is used to implane a fluid membrane on an I.V. assembly so that the radial bulge of the non-sharp vascular infusion cannula passes the membrane. At that time, the radial bulge of the non-sharp vascular infusion cannula holds the non-sharp vascular infusion cannula in place on the I.V. assembly 1503. Next, the user attaches a syringe or other medicine dispenser device to the non-sharp vascular infusion cannula 1504. Medicine is permitted to enter the I.V. assembly from the medicine dispenser through the non-sharp vascular infusion cannula 1505. Next, the medicine dispenser is removed from the non-sharp vascular infusion cannula 1506 while the non-sharp vascular infusion cannula remains held in place at the membrane by the radial bulge. Steps 1504 through 1508 may be repeated as desired 1507 to facilitate multiple instances of medicine being added to an I.V. assembly using a non-sharp vascular infusion cannula and multiple medicine dispensers.

While the present devices and methods have been described and illustrated in conjunction with a number of specific configurations, those skilled in the art will appreciate that variations and modifications may be made without departing from the principles herein illustrated, described, and claimed. The present invention, as defined by the appended claims, may be embodied in other specific forms without departing from its spirit or essential characteristics. The configurations described herein are to be considered in all respects as only illustrative, and not restrictive. All changes which come within the meaning and range of equivalency of the claims are to be embraced within their scope.

I claim:

1. A non-sharp vascular infusion cannula for obtaining a medicine and introducing it to an I.V. assembly to introduction into a patient's body:
a proximal end,
a distal end,
a mechanical connection for attaching to a syringe or other fluid source located at said proximal end,
a non-sharp tip located at said distal end,
an elongate body located between said proximal and distal ends,
a hollow fluid passageway located within the interior of said elongate body, said hollow fluid passageway being capable of permitting the flow of a fluid from said proximal end at least partially to said distal end,
a radial bulge on said elongate body, and
at least one exit orifice located in the vicinity of said distal end, said exit orifice serving to permit fluid to exit said hollow fluid passageway through the exit orifice, or to enter the hollow fluid passageway through the exit orifice.

2. A device as recited in claim 1 wherein said exit orifice is located on said elongate body in a position that causes fluid travelling through the exit orifice to travel at approximately a right angle to fluid travelling through said elongate body.

3. A device as recited in claim 1 wherein said mechanical connection is selected from the group consisting of threaded fittings, luer locks, a tapered receptacle and a friction fit.
wherein the tapered receptacle has tapered cylindrical walls.

4. A device as recited in claim 1 wherein said non-sharp tip has a shape selected from the group consisting of square, blunt, tapered and rounded.

5. A device as recited in claim 1 wherein said non-sharp tip will not impale human skin under the force or ordinary pressure exerted by fingers or hands.

6. A device as recited in claim 1 wherein said exit orifice is located on the side of the non-sharp vascular infusion cannula so that fluid that exits the exit orifice will not be traveling parallel to the path of fluid that is travelling through the hollow fluid passageway in the cannula interior.

7. A device as recited in claim 1 further comprising a one-way valve within the non-sharp vascular infusion cannula so that fluid may only flow in one direction through the cannula.

8. A device as recited in claim 7 wherein said one-way valve permits fluid to flow only from said proximal end to said distal end.

9. A device as recited in claim 7 wherein fluid may only flow from the distal end to the proximal end of the non-sharp vascular infusion cannula, but not the reverse.

10. A device as recited in claim 1 wherein said radial bulge is located closer to said distal end than to said proximal end.

11. A device as recited in claim 1 wherein said radial bulge has a greater radial dimension than said elongate body otherwise has in the vicinity of said elongate body adjacent said radial bulge.

12. A device as recited in claim 1 wherein said radial bulge has the ability to anchor the non-sharp vascular infusion cannula at a membrane of an I.V. apparatus.

13. A device as recited in claim 1 wherein said radial bulge has the ability to anchor the non-sharp vascular infusion cannular at the membrane of a medicine vial.

14. A device as recited in claim 1 wherein said radial bulge has the ability to anchor the non-sharp vascular infusion cannula for repetitive injection or aspiration of medicine.

15. A device as recited in claim 1 wherein if the non-sharp vascular infusion cannula is usable to impale a membrane of a medicine vial or of an I.V. apparatus, and once the radial bulge has penetrated past the membrane, the radial bulge tends to hold the non-sharp vascular infusion cannula in place at the membrane

16. A device as recited in claim 1 wherein said radial bulge has a shape selected from the group consisting of spherical, oval, elliptical, heart shaped, conical, triangular in cross section, rebated at the proximal side in cross section, hooked at the proximal side in cross section, heart shaped in cross section, diamond-shaped in cross section, top-shaped, turnip-shaped, curved and sloped.

17. A device as recited in claim 1 wherein said radial bulge has a steeper curvature or slope on its proximal side than on its distal side to tend to keep the non-sharp vascular infusion cannula in place on a membrane that the non-sharp vascular infusion cannula has impaled past the radial bulge because the radial bulge will tend to make it difficult to withdraw the non-sharp vascular infusion cannula from the membrane.

18. A non-sharp vascular infusion cannula for obtaining a medicine and introducing it to an I.V. assembly to introduction into a patient's body:
a proximal end,
a distal end,
a mechanical connection for attaching to a syringe or other fluid source located at said proximal end,
a non-sharp tip located at said distal end,
an elongate body located between said proximal and distal ends,
a hollow fluid passageway located within the interior of said elongate body, said hollow fluid passageway being capable of permitting the flow of a fluid from said proximal end at least partially to said distal end,
a radial bulge on said elongate body, and
at least one exit orifice located in the vicinity of said distal end, said exit orifice serving to permit fluid to exit said hollow fluid passageway through the exit orifice, or to enter the hollow fluid passageway through the exit orifice.
wherein said exit orifice is located on said elongate body in a position that causes fluid travelling through the exit orifice to travel at approximately a right angle to fluid travelling through said elongate body;
wherein said mechanical connection is selected from the group consisting of threaded fittings, luer locks, a tapered receptacle and a friction fit;
wherein the tapered receptacle has tapered cylindrical walls;
wherein said non-sharp tip has a shape selected from the group consisting of square, blunt, tapered and rounded;
wherein said non-sharp tip will not impale human skin under the force or ordinary pressure exerted by fingers or hands;
wherein said exit orifice is located on the side of the non-sharp vascular infusion cannula so that fluid that exits the exit orifice will not be traveling parallel to the path of fluid that is travelling through the hollow fluid passageway in the cannula interior;
wherein said radial bulge has a greater radial dimension than said elongate body otherwise has in the vicinity of said elongate body adjacent said radial bulge;
wherein said radial bulge has the ability to anchor the non-sharp vascular infusion cannula at a fluid-sealing membrane;
wherein the non-sharp vascular infusion cannula is usable to impale a membrane of a medicine vial or of an I.V. apparatus, and once the radial bulge has penetrated past the membrane, the radial bulge tends to hold the non-sharp vascular infusion cannula in place at the membrane;
wherein said radial bulge has a shape selected from the group consisting of spherical, oval, elliptical, heart shaped, conical, triangular in cross section, rebated at the proximal side in cross section, hooked at the proximal side in cross section, heart shaped in cross section, diamond-shaped in cross section, top-shaped, turnip-shaped, curved and sloped; and
wherein said radial bulge has a steeper curvature or slope on its proximal side than on its distal side to tend to keep the non-sharp vascular infusion cannula in place on a membrane that the non-sharp vascular infusion cannula has impaled past the radial bulge because the radial bulge will tend to make it difficult to withdraw the non-sharp vascular infusion cannula from the membrane.

19. A non-sharp vascular infusion cannula for obtaining a medicine and introducing it to an I.V. assembly to introduction into a patient's body:
a proximal end,
an elongate body,
a fluid passageway at least partially within said elongate body,
a non-sharp tip located at one end of said elongate body,
an exit orifice on said elongate body in the vicinity of said non-sharp tip, said exit orifice serving to permit fluid to exit said fluid passageway to the exterior of said elongate body, and
a radial bulge on said elongate body.

20. A method for aspirating medine from a medicine vial comprising the steps of:
obtaining a syringe,
obtaining a non-sharp vascular infusion cannula that includes
a proximal end,
an elongate body,
a fluid passageway at least partially within said elongate body,
a non-sharp tip located at one end of said elongate body,
an exit orifice on said elongate body in the vicinity of said non-sharp tip, said exit orifice serving to permit fluid to exit said fluid passageway to the exterior of said elongate body, and
a radial bulge on said elongate body,
installing said cannula on said syringe,
selecting a medicine vial from which medicine is to be aspirated,
impaling a fluid-sealing membrane on said medicine vial with said non-sharp tip so that said exit orifice is projected into medicine located within said medicine vial, but so that said radial bulge does not pass said fluid-sealing membrane,
aspirating medicine into said syringe from said medicine vial through said cannula, and
removing said syringe and cannula from said medicine vial.

21. A method for aspirating medine from a medicine vial comprising the steps of:
obtaining a syringe,
obtaining a non-sharp vascular infusion cannula that includes
a proximal end,
an elongate body,
a fluid passageway at least partially within said elongate body,
a non-sharp tip located at one end of said elongate body,
an exit orifice on said elongate body in the vicinity of said non-sharp tip, said exit orifice serving to permit fluid to exit said fluid passageway to the exterior of said elongate body, and
a radial bulge on said elongate body,
installing said cannula on said syringe,
selecting a medicine vial from which medicine is to be aspirated,
impaling a fluid-sealing membrane on said medicine vial with said non-sharp tip so that said exit orifice is projected into medicine located within said medicine vial, and so that said radial bulge passes and projected beyond said fluid-sealing membrane,
aspirating medicine into said syringe from said medicine vial through said cannula,
removing said syringe from said cannula, and
leaving said cannula in place on said medicine vial for later aspiration of additional medicine from said medicine vial.

22. A method for injecting medince from a syringe into an IV apparatus comprising the steps of:
obtaining a non-sharp vascular infusion cannula that includes
a proximal end,
an elongate body,
a fluid passageway at least partially within said elongate body,
a non-sharp tip located at one end of said elongate body,
an exit orifice on said elongate body in the vicinity of said non-sharp tip, said exit orifice serving to permit fluid to exit said fluid passageway to the exterior of said elongate body, and
a radial bulge on said elongate body,
impaling a fluid-sealing membrane on an I.V. appartus with said non-sharp tip so that said radial bulge passes and projected beyond said fluid-sealing membrane,
obtaining a syringe containing a medicine to be injected into the I.V. apparatus,
installing said syringe on said cannula,
injecting medicine from said syringe into the I.V. apparatus through said cannula,
removing said syringe from said cannula, and
leaving said cannula in place on said I.V. apparatus for later injection of additional medicine from into the I.V. apparatus through the cannula.

23. A method for injecting medince from a syringe into an IV apparatus comprising the steps of:
selecting a medicine to injection into an I.V. apparatus,
selecting a syringe to use for said injection,
obtaining a non-sharp vascular infusion cannula that includes
a proximal end,
an elongate body,
a fluid passageway at least partially within said elongate body,
a non-sharp tip located at one end of said elongate body,
an exit orifice on said elongate body in the vicinity of said non-sharp tip, said exit orifice serving to permit fluid to exit said fluid passageway to the exterior of said elongate body, and
a radial bulge on said elongate body,
installing said non-sharp fluid cannula on said syringe,
impaling a fluid-sealing membrane on a medicine vial containing said medicine with said non-sharp tip so that said exit orifice projects into medicine within said vial, and such that said radial bulge does not pass or project beyone said fluid-sealing membrane,
aspirating medicine from said medicine vial into said syringe through said cannula,
removing said cannula from said medicine vial,
impaling a fluid-sealing membrane on an I.V. appartus with said non-sharp tip so that said exit orifice is projected pas said membrane,
into medicine located within said medicine vial, and so that said radial bulge passes and projected beyond said fluid-sealing membrane,
injecting medicine from said syringe into said I.V. apparatus through said cannula, and
removing said syringe and said cannula from said I.V. apparatus.
